(19) United States
(12) Patent Application Publication
(10) Pub. No.: US 2019/0175002 A1
(43) Pub. Date: Jun. 13, 2019

(54) ENDOSCOPE

(71) Applicant: OLYMPUS CORPORATION, Tokyo (JP)

(72) Inventors: Takatoshi IGARASHI, Ina-shi (JP); Kensuke SUGA, Kamiina-gun (JP); Kazuhiro YOSHIDA, Okaya-shi (JP)

(73) Assignee: OLYMPUS CORPORATION, Tokyo (JP)

(21) Appl. No.: 16/281,184

(22) Filed: Feb. 21, 2019

Related U.S. Application Data
(63) Continuation of application No. PCT/JP2016/081897, filed on Oct. 27, 2016.

(51) Int. Cl. A61B 1/05; G02B 23/24; A61B 1/00; A61B 1/12; H04N 5/225

(52) U.S. Cl. CPC A61B 1/05; G02B 23/2476; A61B 1/00096; H01L 23/12; H04N 5/2253; A61B 1/00091; A61B 1/12

(57) ABSTRACT

An endoscope includes: an insertion section including a rigid distal end portion in which a first through hole and a second through hole are formed; an image pickup unit inserted in the first through hole, the image pickup unit including a first block and a second block having an area in a direction orthogonal to an optical axis that is smaller than an area of the first block in the direction orthogonal to the optical axis; and a water feeding and air feeding tube having a distal end portion that is inserted in the second through hole, a part of the tube being arranged in an accommodation space in which a space obtained by extending the first block in an optical axis direction and a space obtained by extending the second block in the direction orthogonal to the optical axis are superimposed on each other.

### CROSS REFERENCE TO RELATED APPLICATION

This application is a continuation application of PCT/JP2016/081897 filed on Oct. 27, 2016, the entire contents of which are incorporated herein by this reference.

### BACKGROUND OF THE INVENTION

### 1. Field of the Invention

The present invention relates to an endoscope including, on a rigid distal end portion, an image pickup unit including a stack of a plurality of semiconductor devices including an image pickup device.

### 2. Description of the Related Art

An endoscope acquires an image of an inside of a body of a patient, for example, by inserting an insertion section having a rigid distal end portion in which an image pickup unit is arranged into the inside of the body. Japanese Patent Application Laid-Open Publication No. 2005-334509 discloses an image pickup unit in which a wiring board on which electronic components such as a capacitor, a resistor, and an IC configuring a drive circuit are mounted is bonded to a rear surface of an image pickup device.

A length of an image pickup unit including a wiring board on which electronic components are mounted in an optical axis direction becomes long. Therefore, it is not easy to cause the rigid distal end portion of the endoscope to be short and small.

In recent years, semiconductor devices in which planar devices (thin film parts) having the same functions as electronic components such as a capacitor are formed have been developed. The image pickup unit can be caused to be short and small by a stack obtained by stacking a plurality of semiconductor devices in which the planar devices are formed with an image pickup device.

### SUMMARY OF THE INVENTION

According to an embodiment of the present invention, there is provided an endoscope including: an insertion section including a rigid distal end portion; and a grip section arranged on a rear portion of the insertion section, in which: a first through hole and a second through hole are formed in the rigid distal end portion; an image pickup unit inserted in the first through hole and including a stack of a plurality of optical elements and a plurality of semiconductor devices including an image pickup device includes: a first block including the image pickup device; and a second block having an area in a direction orthogonal to an optical axis that is smaller than an area of the first block in the direction orthogonal to the optical axis; and a part of a component including a distal end portion inserted in the second through hole is arranged in an accommodation space in which a space obtained by extending the first block in an optical axis direction and a space obtained by extending the second block in the direction orthogonal to the optical axis are superimposed on each other.

### BRIEF DESCRIPTION OF THE DRAWINGS

FIG. 1 is a perspective view of an endoscope of a first embodiment;

FIG. 2 is a perspective view of a rigid distal end portion of the endoscope of the first embodiment;

FIG. 3 is a cross-sectional view of the rigid distal end portion of the endoscope of the first embodiment taken along line of FIG. 2;

FIG. 4 is a cross-sectional view of the rigid distal end portion of the endoscope of the first embodiment taken along line IV-IV of FIG. 3;

FIG. 5 is a cross-sectional view of the rigid distal end portion of the endoscope of the first embodiment taken along line V-V of FIG. 3;

FIG. 6 is a perspective view of an image pickup unit of the endoscope of the first embodiment;

FIG. 7 is a cross-sectional view of a rigid distal end portion of an endoscope of a second embodiment;

FIG. 8 is a cross-sectional view of the rigid distal end portion of the endoscope of the second embodiment taken along line VIII-VIII of FIG. 7;

FIG. 9 is a perspective view of an image pickup unit of an endoscope of a third embodiment; and

FIG. 10 is a front view of a rigid distal end portion of the endoscope of the third embodiment.

### DETAILED DESCRIPTION OF THE PREFERRED EMBODIMENT(S)

### First Embodiment

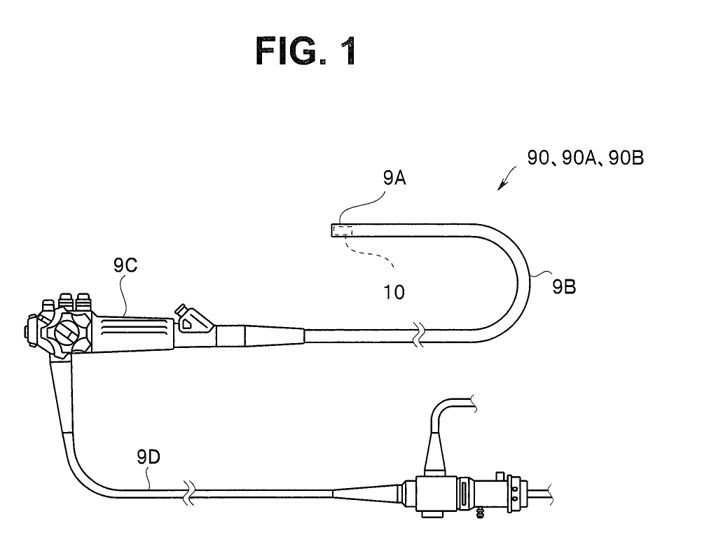

As illustrated in FIG. 1, an endoscope 90 of this embodiment includes an insertion section 9B in which an image pickup unit 10 is accommodated in a rigid distal end portion 9A, a grip section 9C arranged on a rear portion of the insertion section 9B, and a universal cord 9D extending from the grip section 9C. A signal cable 38 connected to the image pickup unit 10 passes through the universal cord 9D.

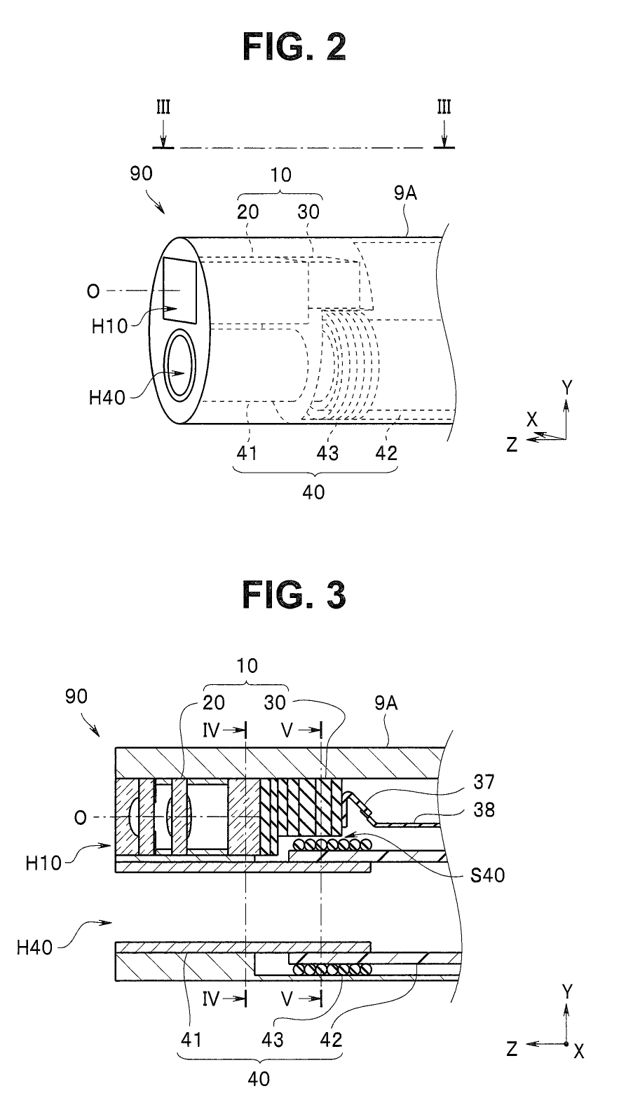

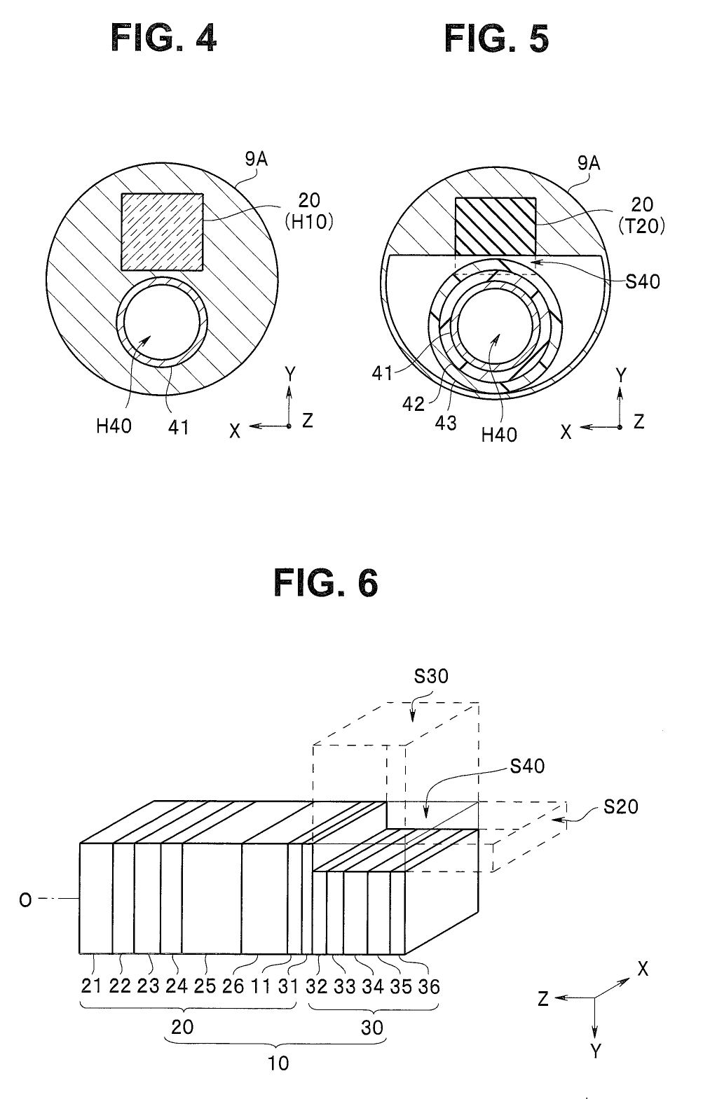

As illustrated in FIG. 2 to FIG. 5, in the rigid distal end portion 9A of the endoscope 90, not only the image pickup unit 10 but also a water feeding and air feeding tube 40 that is a component is arranged. Note that, as described below, the component arranged in the rigid distal end portion 9A is not limited to the water feeding and air feeding tube 40.

Note that, in the following description, the drawings based on each embodiment are schematic, and a relationship between a thickness and a width of each portion, a thickness ratio and a relative angle of each portion, and the like are different from actual relationships, thickness ratios, relative angles, and the like. Portions having different size relationships and ratios may also be included throughout the drawings. Further, drawing of some components may be omitted.

In the optical axis direction, a direction (Z-axis value increasing direction) in which a first block 20 is placed is referred to as a distal end side or a front side, and a direction (Z-axis value decreasing direction) in which a second block 30 is placed is referred to as a rear side.

A first through hole H10 and a second through hole H40 are formed in the rigid distal end portion 9A formed by a metal or a rigid resin. The image pickup unit 10 is inserted in the first through hole, and the water feeding and air feeding tube 40 is inserted in the second through hole H40.

The image pickup unit 10 includes a lens unit including a plurality of optical members 21 to 26, and a stack of a plurality of semiconductor devices 31 to 36 including an image pickup device 11.

Further, as illustrated in FIG. 6, the image pickup unit 10 includes the first block 20 having a rectangular parallelepiped shape and including the lens unit and the image pickup device 11, and the second block 30 having a rectangular parallelepiped shape. The second semiconductor device 32 that is a front surface of the second block 30 is bonded to the first semiconductor device 31 that is a rear surface of the first block 20. The second block 30 is smaller than the first block 20 in terms of an area (a dimension in planar view) in a direction orthogonal to an optical axis. Therefore, an accommodation space S40 in which a space S20 obtained by extending the first block 20 in the optical axis direction and a space S30 obtained by extending the second block 30 in the direction orthogonal to the optical axis are superimposed on each other is provided.

In other words, in the image pickup unit 10, the second block 30 that is smaller than the first block 20 in terms of the dimension in planar view has three side surfaces that are respectively in the same planes as three side surfaces of the first block 20, and has one side surface located on the optical axis side with respect to a side surface of the first block 20. The first block 20 and the second block 30 both have a rectangular shape in planar view, and have the same width (dimension in an X direction) and different heights (dimension in a Y-axis direction). Further, the first block 20 and the second block 30 are bonded to each other so that sides in a height direction overlap with each other.

The water feeding and air feeding tube 40 includes a water feeding pipe 41 that is a first cylindrical member inserted in the second through hole H40, a flexible water feeding tube 42 that is a second cylindrical member put in an outer periphery of a rear end portion of the water feeding pipe 41, and a thread member 43 wound around a connection portion between the water feeding pipe 41 and the water feeding tube 42. In other words, the water feeding tube 42 is connected to a rear portion of the water feeding pipe 41.

The water feeding tube 42 is firmly fixed to the water feeding pipe 41 by fixing the thread member 43 by an adhesive (not shown). A material of the thread member 43 is polyimide, PET (polyethylene terephthalate), or polyvinylidene fluoride, for example. The adhesive is an epoxy resin adhesive, for example. The thread member 43 has a diameter of from 0.05 mm to 0.3 mm, for example.

A part of the thread member 43 and a part of the water feeding tube 42 that are parts of the water feeding and air feeding tube 40, a distal end portion of which is inserted in the second through hole H40, are arranged in the accommodation space S40. In other words, a cross-sectional shape of the water feeding and air feeding tube 40 is circular in the direction orthogonal to the optical axis, and the thread member 43 and the like that are parts of an outer peripheral portion of the water feeding and air feeding tube 40 are arranged in the accommodation space S40.

Now, the accommodation space S40 is a space formed by the configuration of the image pickup unit 10, but a part of the water feeding and air feeding tube 40 that is a component that is not directly related to the image pickup unit 10 is arranged in the accommodation space S40. In other words, the water feeding and air feeding tube 40 is not electrically connected to the image pickup unit 10.

On the rigid distal end portion, components other than the image pickup unit such as a water feeding and air feeding tube are arranged. The water feeding and air feeding tube includes a water feeding pipe inserted in a through hole in the rigid distal end portion, a flexible water feeding tube connected to a rear portion of the water feeding pipe, and a thread member wound around a connection portion between the water feeding pipe and water feeding tube. When the layout of the water feeding and air feeding tube in the rigid distal end portion is designed in accordance with a winding portion having a largest outer diameter, an outer diameter of the rigid distal end portion becomes large.

However, a part of the water feeding and air feeding tube 40 is arranged in the accommodation space S40, and hence the rigid distal end portion 9A of the endoscope 90 is small in diameter and minimally invasive.

For example, in an endoscope in which the outer diameter of the rigid distal end portion 9A is about 3 mm, the outer diameter of the outer diameter of the rigid distal end portion 9A can be reduced by from about 0.1 mm to about 0.5 mm by the configuration of the present invention.

Next, components of the endoscope 90 are described in detail.

The first block 20 of the image pickup unit 10 includes a concave lens 21 on the distal end, a transparent plate 22 in which an aperture is arranged, a spacer 23, a transparent plate 24 configuring a convex lens, a spacer 25, a cover glass 26, the image pickup device 11, and the first semiconductor device 31.

Light that has entered the image pickup device 11 that is a CCD or a CMOS light receiving element via the concave lens 21 to the cover glass 26 is converted to an image pickup signal through photoelectric conversion, and the image pickup signal is transmitted to an electrode on the rear surface via through wiring (not shown). The image pickup device 11 and the first semiconductor device 31 are connected to each other via bonding portions such as bumps (not shown).

The second block 30 of the image pickup unit 10 includes the second semiconductor device 32, the third semiconductor device 33, the fourth semiconductor device 34, the fifth semiconductor device 35, and the sixth semiconductor device 36. The second semiconductor device 32 to the sixth semiconductor device 36 are also connected via wiring and bumps (not shown). Note that spaces between the devices are sealed with sealing resin layers (underfilling). The sealing resin layer is formed by an insulating resin such as an epoxy resin, an acrylic resin, a polyimide resin, a silicone resin, or a polyvinyl resin.

In each of the first semiconductor device 31 to the sixth semiconductor device 36, a planar device configuring an electronic component function circuit such as a capacitor, a resistor, or a buffer, or a signal processing circuit such as a noise reduction circuit or an analog-to-digital converter circuit is formed. The signal cable 38 is connected to a rear surface of the sixth semiconductor device 36 via a wiring board 37.

A thickness of each of the semiconductor devices 31 to 36 is from about 30 μm to about 100 μm. The planar device may be formed on one surface or both surfaces of each of the semiconductor devices 31 to 36. The number of the semiconductor devices 31 to 36 only needs to be two or more, and is not limited to six as in this example.

The first block 20 and the second block 30 are so-called wafer-level structures. For example, the first block 20 is manufactured by cutting stacked wafers obtained by bonding a plurality of optical wafers respectively including a plurality of optical members 21 to 26, an image pickup device wafer including a plurality of the image pickup devices 11, and a plurality of semiconductor wafers including a plurality of the first semiconductor devices 31. The first block 20 and the second block 30 having different areas in the direction orthogonal to the optical axis are bonded to each other after the respective stacked wafers are cut.

Needless to say, although the productivity is not good, the first block 20 and the like may be manufactured by bonding a plurality of elements after cutting an element wafer including the plurality of elements. In contrast, the accommodation space S40 may be manufactured by manufacturing stacked wafers in which all of the wafers are stacked, and grinding a region in which the planar device and the like are not formed through so-called step-cut dicing. The accommodation space S40 may also be manufactured by removing a region in which the planar device and the like are not formed by etching.

Note that the first block 20 only needs to include the image pickup device 11, and the first semiconductor device 31 may be included in the second block 30. In contrast, a plurality of semiconductor devices may be included in the first block 20. Further, the second block 30 only needs to include at least one semiconductor device.

As illustrated in FIG. 5, in the rigid distal end portion 9A of the endoscope 90, a rear portion of the first through hole H10 that is located behind a rear opening of the second through hole H40 is a groove T20, one surface of which is an opening. Further, a part of the groove T20 configures the accommodation space S40. Therefore, by inserting the water feeding and air feeding tube 40 in the second through hole H40 after inserting the image pickup unit 10 in the first through hole H10 and the groove T20, the thread member 43 having a large outer diameter can be arranged in the accommodation space S40. Therefore, the endoscope 90 is easy to manufacture.

### Second Embodiment

An endoscope 90A of a second embodiment is similar to and has the same effect as the endoscope 90, and hence components having the same functions are denoted by the same reference characters and description of those components are omitted.

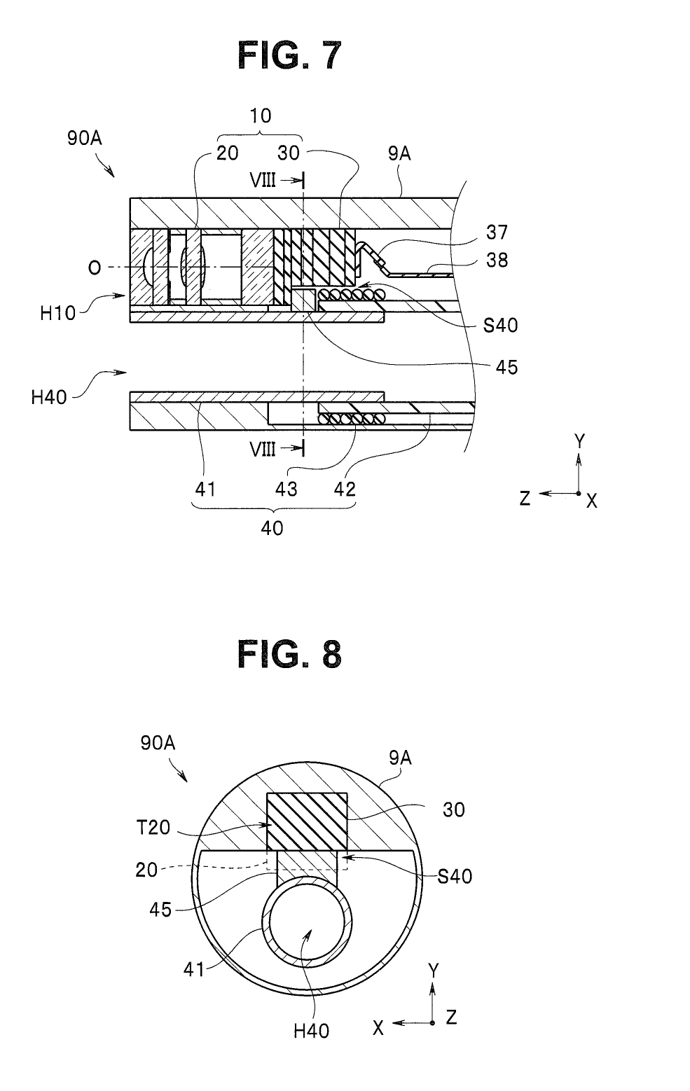

As illustrated in FIG. 7 and FIG. 8, in the endoscope 90A, a metal member 45 made of copper, for example, is arranged on the water feeding pipe 41 made of metal of the water feeding and air feeding tube 40 that is a component. The metal member 45 is in abutment against the rear surface of the first block 20. In other words, the endoscope 90A is configured so that heat generated by the image pickup unit 10 is transferred to the water feeding and air feeding tube 40 via the metal member 45.

An abutting surface between the metal member 45 and the rear surface of the first block 20 is large, and hence heat can be efficiently transferred. Note that, instead of the metal member 45 made of copper, a highly heat-conductive member formed by a highly heat-conductive material such as aluminum, silicon, AIN, or a graphite-metal composite material may be used.

### Third Embodiment

An endoscope 90B of a third embodiment is similar to and has the same effect as the endoscope 90, and hence components having the same functions are denoted by the same reference characters and description of the components is omitted.

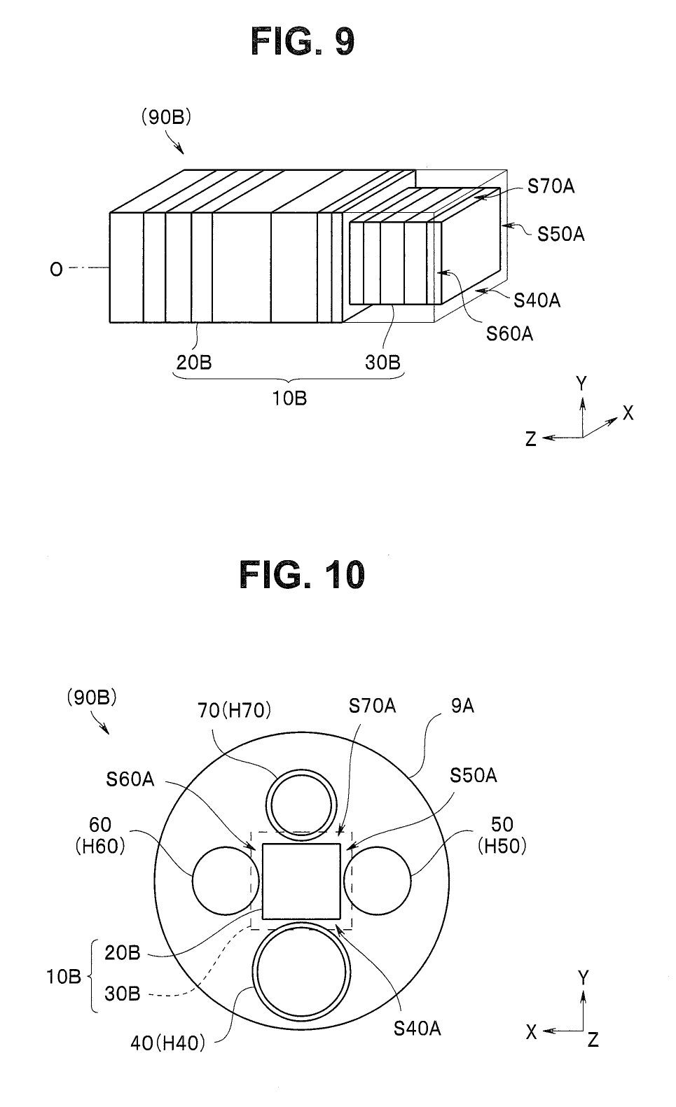

As illustrated in FIG. 9, in an image pickup unit 10B of the endoscope 90B, there are four accommodation spaces S40A, S50A, S60A, and S70A on a periphery of a second block 30B. In other words, in the image pickup unit 10B, the second block 30B having a smaller dimension in planar view as compared to a first block 20B is bonded to approximately a center of a rear surface of the first block 20B. Further, as illustrated in FIG. 10, five through holes 1110, H40, H50, 1160, and H70 are formed in the rigid distal end portion 9A.

The image pickup unit 10B is inserted in the through hole H10 in the center having a rectangular shape in planar view. The water feeding and air feeding tube 40 is inserted in the through hole 1140 having a circular shape in planar view. Light guides 50 and 60 are respectively inserted in the through holes H50 and H60 having a circular shape in planar view. A treatment instrument channel 70 is inserted in the through hole H70 having a circular shape in planar view.

In each of the light guides 50 and 60, a plurality of thin optical fibers are bundled, and an outer periphery is covered with a covering. The covering is removed from distal end portions inserted in the through holes H50 and H60. On a rear portion of the distal end portion, the thread member is wound around the plurality of optical fibers. A part of the thread member is arranged in the accommodation spaces S50A and S60A.

The treatment instrument channel 70 inserted in the through hole H70 has approximately the same configuration as the water feeding and air feeding tube 40, and includes a distal end pipe, a flexible channel tube, and a thread member wound around a connection portion. A part of the thread member is arranged in the accommodation space S70A.

As described above, in the endoscope 90B, at least the third through hole H50 is further formed in the rigid distal end portion 9A, and a part of the light guide 50, which is a second component and has a distal end portion that is inserted in the third through hole H50, is arranged in the accommodation space S50A.

Needless to say, the second component may be a treatment instrument channel in the endoscopes 90 and 90A, as well. A component having a distal end portion that is inserted in the through hole H40 may be the treatment instrument channel. Further, as in the endoscope 90B, four or more through holes may be formed in the rigid distal end portion 9A, or a plurality of components may have the same configuration. Accommodation spaces may be above two side surfaces or above three side surfaces of the second block.

In the first to third embodiments, all the components having a distal end portion inserted in a through hole have a circular shape in terms of a cross-sectional shape (shape in planar view) of an outer peripheral surface in the direction orthogonal to the optical axis, but the components may be members having a rectangular shape, a polygonal shape, or the like. A component arranged in an accommodation space may be a member protruding from a part of the outer periphery of a component as with the metal member 45.

The endoscope 90 and the like are soft endoscopes, but may be rigid endoscopes, and may be medical endoscopes or industrial endoscopes.

The present invention is not limited to the abovementioned embodiments, and various modifications, changes, and the like can be made within the range in which the gist of the present invention is not changed.

What is claimed is:

1. An endoscope, comprising:
an insertion section comprising a rigid distal end portion; and
a grip section arranged on a rear portion of the insertion section, wherein:
a first through hole and a second through hole are formed in the rigid distal end portion;
an image pickup unit inserted in the first through hole and comprising a stack of a plurality of optical elements and a plurality of semiconductor devices comprising an image pickup device comprises:
a first block comprising the image pickup device; and
a second block having an area in a direction orthogonal to an optical axis that is smaller than an area of the first block in the direction orthogonal to the optical axis; and
a part of a component including a distal end portion inserted in the second through hole is arranged in an accommodation space in which a space obtained by extending the first block in an optical axis direction and a space obtained by extending the second block in the direction orthogonal to the optical axis are superimposed on each other.

2. The endoscope according to claim 1, wherein:
the component inserted in the second through hole has a circular cross-sectional shape in the direction orthogonal to the optical axis; and
a part of an outer peripheral portion of the component is arranged in the accommodation space.

3. The endoscope according to claim 2, wherein:
the component comprises:
a first cylindrical member inserted in the second through hole;
a second cylindrical member connected to a rear portion of the first cylindrical member; and
a thread member wound around a connection portion between the first cylindrical member and the second cylindrical member; and
a part of the thread member is arranged in the accommodation space.

4. The endoscope according to claim 1, wherein:
a third through hole is further formed in the rigid distal end portion; and
a part of a second component including a distal end portion inserted in the third through hole is arranged in a second accommodation space in which a space obtained by extending the first block in the optical axis direction and a space obtained by extending the second block in the direction orthogonal to the optical axis are superimposed on each other.

5. The endoscope according to claim 1, wherein the component is at least one of a water feeding and air feeding tube, a treatment instrument channel, and a light guide.

6. The endoscope according to claim 1, wherein:
a metal member abutting against a rear surface of the first block is arranged in the component; and
heat generated by the image pickup unit is transferred to the component via the metal member.
